(19) United States
(12) Patent Application Publication
(10) Pub. No.: US 2010/0094134 A1
(43) Pub. Date: Apr. 15, 2010

(54) METHOD AND APPARATUS FOR MEDICAL IMAGING USING NEAR-INFRARED OPTICAL TOMOGRAPHY COMBINED WITH PHOTOACOUSTIC AND ULTRASOUND GUIDANCE

(75) Inventors: Quing Zhu, Mansfield, CT (US); John Gamelin, Avon, CT (US)

Correspondence Address: CANTOR COLBURN, LLP, 20 Church Street, 22nd Floor, Hartford, CT, 06103, US

(73) Assignee: THE UNIVERSITY OF CONNECTICUT, Farmington, CT (US)

(21) Appl. No.: 12/578,799

(22) Filed: Oct. 14, 2009

Related U.S. Application Data
(60) Provisional application No. 61/105,174, filed on Oct. 14, 2008.

(51) Int. Cl. A61B 6/00

(52) U.S. Cl. 600/473

(57) ABSTRACT

Disclosed herein is an apparatus for biological imaging comprising a probe comprising an emitter and a detector; a source circuit connected in operational communication to the emitter; a detector circuit connected in operational communication to the detector; a central processing unit connected to the source circuit and the detector circuit; a display operably connected to the central processing unit; and wherein the apparatus is capable of photoacoustic tomography and diffusive optical tomography and/or ultrasound tomography.

### CROSS-REFERENCE TO RELATED APPLICATION

This application claims priority to provisional application 61/105,174 filed on Oct. 14, 2009, the entire contents of which are hereby incorporated by reference.

### TECHNICAL FIELD

This disclosure relates primarily to the field of biological imaging, particularly to medical imaging. More specifically, this disclosure relates to medical imaging equipment and methods for medical imaging using combined near-infrared optical tomography with photoacoustic and ultrasound guidance.

### BACKGROUND OF THE INVENTION

Diffusive optical tomography (DOT) is a form of computer-generated tomography wherein near-infrared light (NIR) is directed at a biological object (e.g., a inclusion, tumor, and so forth) and the amount of light transmitted and/or diffused through the object, and/or reflected from the object, is detected and utilized to reconstruct a digital image of the target area (e.g., the object can exhibit a differential in optical absorption and light scattering from surrounding tissues). This method of imaging is of interest for several reasons. For example, differing soft tissues exhibit differing absorption and scattering of near-infrared light. Therefore, DOT is capable of differentiating between various types of tissues, where alternative tomography methods (e.g., Positron Emission Tomography, Magnetic Resonance Imaging, X-Ray, and so forth) have difficulty. Another advantage is that near-infrared light is non-ionizing to body tissue and as a result patients can be subjected to repeated light illumination without harm. This in turn allows physicians to increase the frequency at which they monitor and/or track change in areas of interest (e.g., inclusions or tumors present in the breast and so forth). In addition, due to the differences at which natural chromophores (e.g., oxygen-hemoglobin, deoxygenated-hemoglobin) absorb light, optical tomography is capable of supplying functional information such as hemoglobin concentration. For these reasons there is much interest in employing optical tomography for the detection and monitoring of soft tissues, especially in breast cancer applications.

Although diffusive optical tomography is a promising medical imaging technique, DOT imaging methods have yet to yield high quality reconstructions of inclusions due to fundamental issues with intense light scattering. The primary limitation of DOT is related to the intense light scattering in tissues that dominates near infrared light propagation and makes three-dimensional localization of lesions and accurate quantification of lesion optical properties difficult. In order to circumvent these difficulties with DOT, it is often combined (also termed co-registration) with ultrasound imaging. In this co-registered method, a larger region of interest containing a suspicious lesion is detected by another modality is used to guide DOT image reconstruction.

Ultrasound imaging is a well-developed medical diagnostic that is used extensively for differentiation of cysts from solid lesions in breast examinations, and it is routinely used in conjunction with mammography to differentiate simple cysts from solid lesions. Ultrasound can detect breast lesions that are a few millimeters in size; however, its specificity in breast cancer detection is not high as a result of the overlapping characteristics of benign and malignant lesions. The sonographic appearances of benign and malignant lesions have considerable overlapping features, which has prompted many radiologists to recommend biopsies on most solid nodules. Thus, the insufficient specificity provided by ultrasound results in a large number of biopsies yielding benign breast masses or benign breast tissue (currently 70 to 80 percent of biopsies yield benign changes). In addition, due to the different contrast mechanisms between ultrasound imaging and DOT, some lesions that have high optical contrast may not be detected with a non-optical modality such as ultrasound. In the presence of multiple targets within the region of interest, discrimination of true absorptive features becomes virtually impossible without guidance based on multiple forms of optical contrast.

### SUMMARY

Disclosed herein is a method for medical imaging comprising scanning a tissue volume with near-infrared photoacoustic laser beam to obtain a first set of structural parameters, wherein the tissue volume includes a biological entity; receiving from the tissue an acoustic signal in response to scanning the tissue volume with the laser beam; the acoustic signal being processed to obtain a first set of structural parameters; scanning the tissue with ultrasonic waves to obtain a second set of structural parameters; scanning the tissue with near-infrared diffusive light to obtain a third set of structural parameters; and processing the first and second sets of structural parameters and localizing the biological entity using these parameters to quantitatively reconstruct the functional parameters of the biological entity from the third set of structural parameters.

Disclosed herein is an apparatus for biological imaging comprising a probe comprising an emitter and a detector; a source circuit connected in operational communication to the emitter; a detector circuit connected in operational communication to the detector; a central processing unit connected to the source circuit and the detector circuit; a display operably connected to the central processing unit; and wherein the apparatus wherein the apparatus is operative to perform photoacoustic tomography and diffusive optical tomography and/or ultrasound tomography.

Disclosed herein too is a probe comprising a faceplate having a first surface and a second surface; the first surface being opposed to the second surface; an ultrasound transducer; the ultrasound transducer being disposed in the faceplate and having a surface that is parallel to the first surface of the faceplate; a perimeter of ultrasound transducer being surrounded by light absorbing material; the faceplate having openings for accommodating a plurality of first emitters and second emitters; first emitters; and second emitters; wherein the first emitters are closer to a center of the faceplate than the second emitters.

The above described and other features are exemplified by the following figures and detailed description.

### BRIEF DESCRIPTION OF THE DRAWINGS

Referring now to the figures, which are exemplary embodiments, and wherein the like elements are numbered alike.

FIG. 1 is a simplified block diagram of an exemplary imaging system;

FIG. 2 is a photographic image of an exemplary prototype handheld probe;

FIG. 3 is a diagram of the exemplary hand-held probe;

FIG. 4 is a front view of an exemplary faceplate of the probe when the probe is used in a reflection mode;

FIG. 5 is a front view of an exemplary faceplate of the probe when the probe is used in an orthogonal mode; system; probe;

FIG. 6 is a depiction of an exemplary faceplate where the photoacoustic fibers are closer to the ultrasound transducer than the DOT source fibers or the DOT detector fibers;

FIG. 7 is a depiction of exemplary circuitry used in the medical imaging apparatus;

FIG. 8 depicts a picture of the experimental configuration for the orthogonal DOT/PAT geometry;

FIG. 9A depicts an exemplary probe that is used in the orthogonal mode, while FIG. 9B depicts an exemplary probe that is used in the reflection mode;

FIG. 10A depicts images for the two resin balls at a separation of 2.5 cm at a depth of 1.5 cm; while FIG. 10B depicts images using depth-only guidance and FIG. 10C depicts images using PAT guidance;

FIG. 11 depicts PAT images of the two targets located at 1.0, 1.5, and 2.0 cm depths from left to right with approximately 2.0 cm target separation;

FIG. 12A depicts photographic images that show very low absorbing silicone targets at almost 2 cm depth. FIG. 12B shows DOT images using the full PAT information that permits quantification to 78% (b, top). FIG. 12C shows that because the actual target was slightly offset from the center of the defined mesh, quantification was reduced to 43% and the resulting images were not well defined (C, bottom);

FIG. 13 is a photograph of the faceplate of the probe used in the Example 2;

FIG. 14 is the ultrasound transducer frequency response;

FIG. 15 depicts the profiles for absorbing (Reff=0, black color absorbing surface), partial absorbing (Reff=0.4, gray or red color absorbing surface), and partial reflecting (Reff=0.6, white color reflecting surface) probe surfaces;

FIG. 16 shows the improvement in both DOT localization and quantification provided with PAT guidance. FIG. 16A is a photograph showing depth of the absorber versus lateral position traversed across the surface with the probe. FIG. 16B shows photographs that depict the image obtained using only the DOT probe for the single-lobed inclusion. FIG. 16C shows photographs that depict the image obtained using both the DOT and the PAT probe for the single-lobed inclusion;

FIG. 17 represents the reconstructed absorption values of one high-contrast target (tumor like) and one low-contrast target (benign lesion) versus depth; The dashed lines were true values and solid line represents the reconstructed absorption of the high-contrast target at a different depth and blue line shows the reconstructed absorption of the low-contrast target at a different depth; and

FIG. 18 shows the reconstructed absorbers with and without PAT guidance for a multi-lobed absorber. FIG. 18A is a photograph showing depth of the absorber versus lateral position traversed across the surface with the probe. FIG. 18B shows photographs that depict the image obtained using only the DOT probe for the multi-lobed inclusion. FIG. 18C shows photographs that depict the image obtained using both the DOT and the PAT probe for the multi-lobed inclusion.

### DETAILED DESCRIPTION OF THE INVENTION

It is to be noted that as used herein, the terms “first,” “second,” and the like do not denote any order or importance, but rather are used to distinguish one element from another, and the terms “the”, “a” and “an” do not denote a limitation of quantity, but rather denote the presence of at least one of the referenced item. Furthermore, all ranges disclosed herein are inclusive of the endpoints and independently combinable.

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The transition phrase “comprising” is inclusive of the transition phrases “consisting essentially of” and “consisting of”.

At the outset, it is to be understood that the term inclusion is to be interpreted as any tissue(s), biological mass(es), biological entity(ies), and/or foreign object(s) that can be differentiated from surrounding tissue(s), biological mass(es), and/or biological entity(ies), using diffusive optical tomography (DOT), Ultrasound tomography (US) and/or photoacoustic tomography (PAT). For example, an inclusion can be a tumor that is disposed within soft tissues, such as a tumor within a female breast, wherein the tumor (e.g., comprising epithelial tissues, masenchymal tissues, and so forth) exhibits dissimilar optical and/or physical properties from the surrounding tissues. Also, the term inclusion as used herein can be used interchangeably with the terms biological entity and target. Further, the term structural information refers to any information gathered or determined with respect to the structure, or physical shape, of an inclusion, such as position (e.g., X, Y, Z coordinates), diameter, mass, volume, shape (e.g., circular, elliptical, and so forth), and so forth. Lastly, the term “functional information” is to be interpreted as any information gathered or determined that can be employed by a physician, operator, or one skilled in the art, to determine additional characteristics about the inclusion.

Disclosed herein is a medical imaging apparatus and methods for medical imaging wherein diffuse optical tomography (DOT) is used in conjunction with photoacoustic tomography (PAT) and/or ultrasound (US) to detect lesions and tumors. Photoacoustically obtained high-resolution microvessel maps guide the diffuse optical tomography quantitative imaging reconstruction. Compared with the use of ultrasound guidance that only probes tumor mechanical contrast, photoacoustic guidance provides a qualitative map of tumor optical contrast to precisely guide the accurate reconstruction of tumor vasculature obtained from diffuse optical tomography as a result of which there is an improved diagnosis.

Photoacoustic tomography (PAT) is an emerging technique, in which a short-pulsed laser beam penetrates into the tissue sample diffusively. Thermoelastic expansion resulting from a transient temperature rise, caused by the laser irradiation, generates photoacoustic waves, which are then measured by ultrasound transducers. The acquired photoacoustic waves are used to reconstruct, at ultrasound resolution, the optical absorption distribution that reveals optical contrast. However, the robustness of optical property quantification by PAT is complicated because of the wide range of US transducer bandwidth and sensitivity, the orientation and shape of the targets relative to the US receiving aperture, and the uniformity of the laser beam. Currently, several groups are investigating quantitative PAT using Finite Element light diffusion forward models and phantoms of known optical properties to calibrate the sensitivity of the US transducer. However, the studies are limited to simple phantoms and the clinical viability remains to be demonstrated. In this application, we present a novel PAT-guided DOT approach that utilizes qualitative or relative target absorption maps detected by PAT to guide the selection of multiple ROIs for quantitative DOT image reconstruction of optical properties of multiple targets. The PAT guidance can be combined with ultrasound guidance to DOT. This hybrid approach combines the advantages of PAT and/or ultrasound and DOT and has a great potential to provide optical detection and characterization of deeply seated tumors.

In one embodiment, the method comprises scanning a tissue volume with photoacoustic laser light to obtain photoacoustic signals. The photoacoustic signal is processed to obtain a first set of structural parameters. The tissue volume is then scanned with ultrasonic waves to obtain a second set of structural parameters. The scanned tissue is then scanned with a near-infrared diffusive light beam to obtain functional parameters of the biological entity using the guidance of the first and/or second set of structural parameters about the location and size of the biological entity.

In another embodiment, the method for medical imaging comprises scanning the tissue volume with near-infrared photoacoustic light beam; receiving from the tissue the laser-beam-generated acoustic or photoacoustic signal in response to scanning the tissue volume with the laser beam; the photoacoustic signal being processed to obtain a first set of structural parameters. The first set of structural parameters are related to optical contrast of a biological entity within the tissue volume. The tissue is then scanned with ultrasonic waves to obtain a second set of structural parameters related to acoustic contrast of the biological entity. The data obtained from the photo acoustic signal and the ultrasonic waves are then mathematically processed to get the first and second sets of structural parameters to obtain information about the location, size, acoustic contrast, and qualitative optical contrast of the biological entity.

A third set of data (e.g., structural parameters) can be obtained by scanning the tissue volume with near infrared diffusive light beam to obtain functional parameters related to quantitative optical contrast of the biological entity. This third set of data is mathematically processed using the structural information obtained from the first and second sets of data to obtain optical absorption, hemoglobin concentration (deoxygenated hemoglobin, oxygenated hemoglobin and total hemoglobin), and oxygen saturation or other optically probed parameters of the biological entity.

In one embodiment, the diffuse optical tomography can be employed in conjunction with only photoacoustic tomography. In another embodiment, the diffuse optical tomography can be employed in conjunction with photoacoustic tomography as well as with ultrasound. In yet another embodiment, the diffuse optical tomography can include near infrared diffuse optical tomography. Diffuse optical tomography (DOT) in the near infrared region (NIR) provides a unique approach for functional based diagnostic imaging. However, the intense light scattering in tissue produced by the DOT dominates the NIR light propagation regime and makes three-dimensional localization of lesions and accurate quantification of lesion optical properties difficult. Optical tomography guided by co-registered photoacoustic tomography and/or ultrasound has a great potential to overcome lesion location uncertainty and to improve light quantification accuracy.

Photoacoustic tomography can be used to probe tumors that are about 2 to about 3 centimeters under the skin. However, ultrasound and diffuse optical tomography are capable of probing up to about 4 to about 5 centimeters depth in biological tissue. The system disclosed herein has the ultrasound probe working in dual mode for a) receiving photoacoustic signals for photoacoustic high-resolution imaging and precise guidance for quantitative DOT imaging, especially for lesions up to about 2 to about 3 centimeters depth (most common in breast imaging); and b) pure ultrasound pulse-echo imaging to provide tumor mechanical contrast and guiding diffuse optical tomography to image deeply seated tumors and/or provide multi-modality validation of possible tumor locations and size. The disclosed hybrid system thus has three imaging modalities integrated into one hand-held probe for cancer detection and imaging.

The use of three imaging modalities can be used to detect differences in biological tissues up to a depth of about 0.5 centimeters to about 10 centimeters, specifically about 1 to about 5 centimeters, and more specifically about 2 to about 4 centimeters.

The robustness of optical property quantification by only PAT is generally complicated due to several factors: the ultra-wideband photoacoustic frequency response, dependencies upon the orientation, size, and shape of the targets with respect to the ultrasound receiving aperture, uniformity of the light illumination, and uncertainty in optical parameters such as scattering not provided by the photoacoustic measurement. The disclosed hybrid system significantly improves cancer detection and imaging. The disclosed system employs a novel PAT-guided DOT approach that utilizes qualitative or relative target absorption maps detected by PAT to guide the selection of multiple regions of interest (ROI) for quantitative DOT image reconstruction of optical properties for one or more targets. This hybrid approach combines the advantages of PAT and DOT and has a great potential to provide optical detection and characterization of deep-seated tumors.

Photoacoustic tomography involves the irradiation of an area of tissue with a source of light. The source of light is generally a laser. The laser can be dye laser, a solid-state laser and/or a diode laser. In addition, the laser may be pulsed or continuously modulated and have a wide range of wavelengths.

The wavelengths used of the laser light used for photoacoustic tomography is about 600 to about 1,500 nanometers, specifically about 650 to about 1,000 nanometers. A preferred wavelength is about 770 nanometers. The wavelengths of the near infrared light used for DOT are about 600 nanometers to about 100 micrometers, specifically about 630 nanometers to about 1,500 nanometers, and more specifically about 660 nanometers to about 1,000 nanometers.

The energy absorbed by the tissue from the laser is transformed into kinetic energy by an energy exchange process. This results in local heating. The local heating of the tissue produces expansion as a result of which a pressure wave or a sound wave is generated. By measuring the sound wave at different optical wavelengths, a photoacoustic spectrum of the tissue can be recorded that can be used to identify the absorbing components of the tissue.

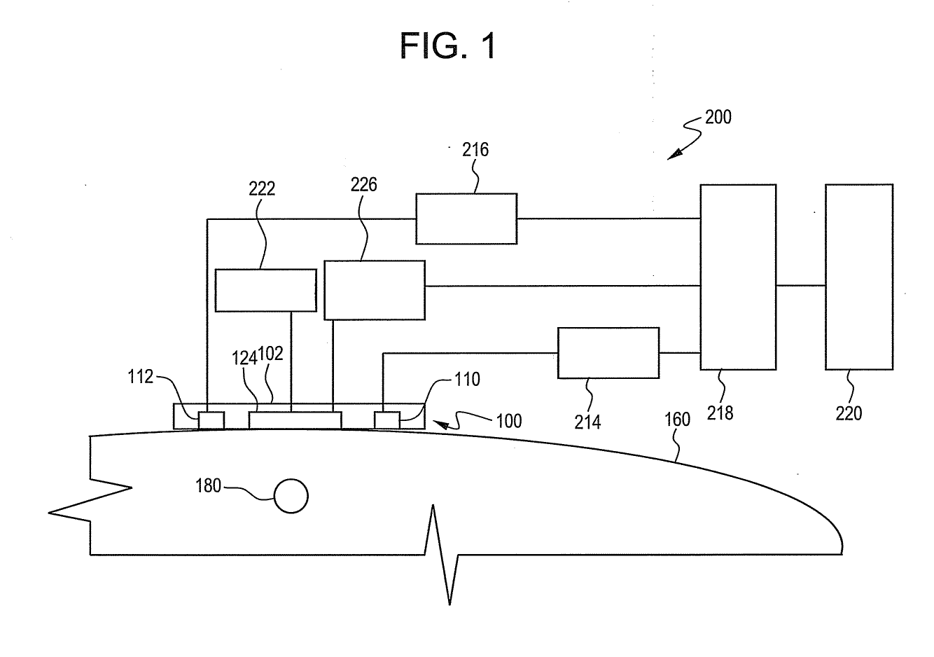

Referring now to FIG. 1, a simplified block diagram of an imaging system 200 is illustrated, wherein the imaging system 200 comprises a probe 100 that can be disposed on bodily tissue 160 to image an inclusion 180 therein. The probe 100 comprises a first emitter 110 and a first detector 112, wherein the emitter 110 is connected in operational communication to a source circuit 214, and the detector 112 is connected in operational communication to a detector circuit 216. The source circuit 214 and detector circuit 216 are operably connected to a central processing unit 218 (hereinafter referred to as CPU 218), which is operably connected to a display 220 on which an image of the inclusion 180 can be generated. The CPU 218 is capable of controlling the operation of the imaging system 200.

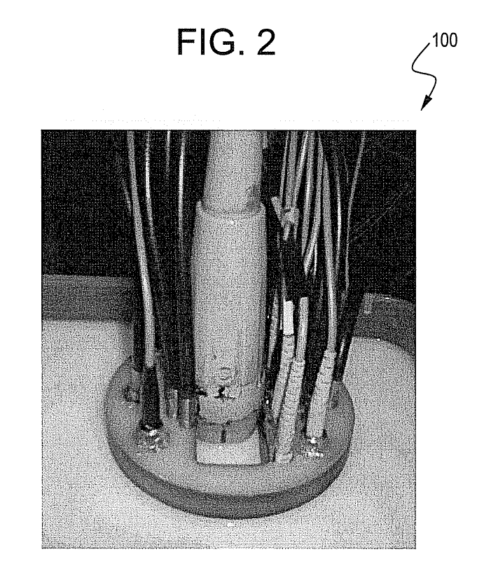

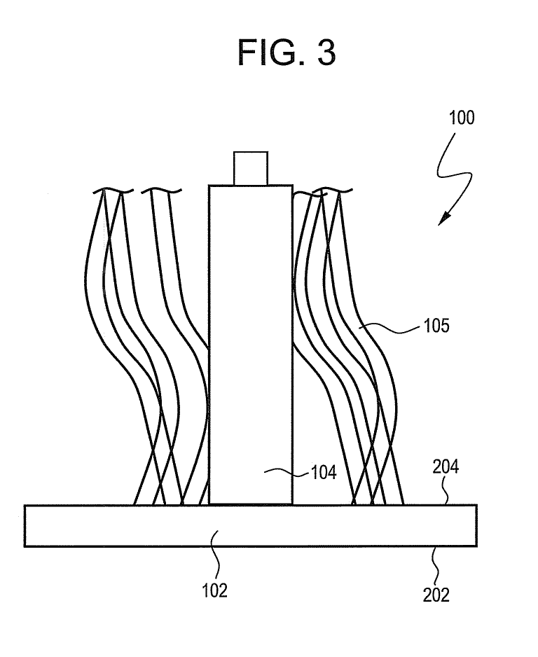

FIG. 2 depicts photograph of a hand-held hybrid reflection-geometry probe. FIG. 3 is a schematic diagram of a side view of the hand-held hybrid reflection-geometry probe 100 (hereinafter probe 100) having a faceplate 102 that contacts the patient. As can be seen in the FIGS. 1, 2 and 3 there are a number of cables 105 that carry optical, acoustical and electrical signals from generators to the probe 100 and from the probe 100 to detector circuit 216. An ultrasound transducer 104 is disposed on the side of the probe 100 that is opposed to the faceplate 102. The FIG. 3 is only a schematic diagram and does not contain an accurate number of cables as shown in the photograph in the FIG. 2.

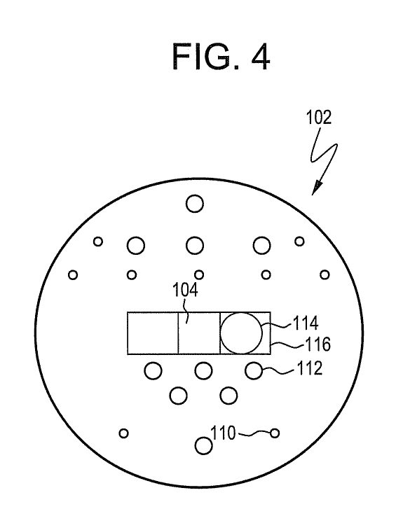

FIG. 4 is a front view of one exemplary embodiment of the faceplate 102 of the probe 100. In this exemplary embodiment, the probe 100 operates in the reflection mode. The probe comprises a plurality of first emitters 110 and first detectors 112 disposed on a faceplate 102. In another embodiment, the probe comprises a plurality of second emitters and a plurality of second detectors (not shown). In the embodiment depicted in the FIG. 4, an ultrasound transducer 104 is disposed on the faceplate 102 of the probe 100. The faceplate 102 comprises a first surface 202 and a second surface 204 that is opposed to the first surface 102. The first surface 202 generally contacts the surface of a patient to detect inclusions.

The center of the ultrasound transducer 104 is located near the center of the faceplate 102. In one embodiment, the center of the ultrasound transducer 104 is the center of the faceplate 102 (i.e., they are concentrically situated). In another embodiment, the center of the ultrasound transducer is not at the center of the faceplate 102 (i.e., the ultrasound transducer 104 is eccentrically situated with respect to the faceplate 102).

The ultrasound transducer 104 is disposed in the faceplate and has a surface that is parallel to the first surface of the faceplate. In one embodiment, the surface of the ultrasound transducer 104 may not be parallel to the first surface 202 of the faceplate 102.

The faceplate 102 of the probe 100 also has disposed upon its surface an opening 114 through which a photoacoustic laser light may be disposed upon the surface of the patient. The laser light entering the opening is generally inclined at an angle of about 20 to about 75 degrees, specifically about 30 to about 45 degrees with respect to a line that is perpendicular to a surface of the faceplate that contacts the patient. The opening has a diameter of about 1 to about 4 centimeters, specifically about 2 centimeters. The laser light is absorbed by the lesions or tumors in the patients and the optical-absorption-generated acoustic waves are detected by the ultrasound transducer. The photoacoustic laser light is not always emitted through the opening 114. In one embodiment, the laser light may be coupled to the probe through optical fibers 105 (FIG. 3) and detected by the ultrasound transducer 104.

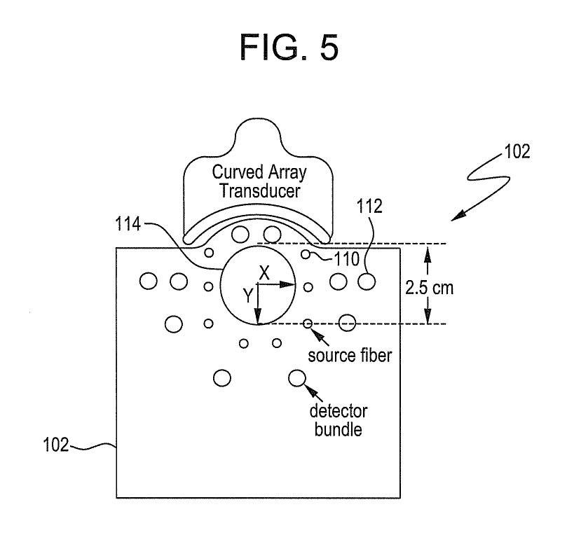

For the diffusive light radiation, the first emitters 110 are capable of emitting near infrared light. The first detectors 112 are capable of detecting radiation emitted by the emitters 110. Any number of first emitters 110 and first detectors 112 can be employed to perform the functional imaging. In an exemplary embodiment, the probe 100 can comprise about 1 to about 30 first emitters, specifically about 2 to about 20 first emitters and more specifically about 5 to about 10 first emitters. A preferred number of first emitters in the probe 100 is about 9. In another exemplary embodiment, the probe 100 can comprise about 1 to about 30 first detectors, specifically about 2 to about 20 first detectors and more specifically about 10 to about 14 first detectors. A preferred number of first detectors in the probe 100 is about 10 or 14. It is noted however that as the number of first emitters 110 and/or first detectors 112 increases, the imaging time (e.g., the time elapsed before radiation received by a detector can be processed and reconstructed into an image and displayed on display 220 by CPU 218, shown in the FIG. 5) can increase due to the additional information to be processed by the CPU 218.

The first emitters 110 and the first detectors 112 are generally disposed on the surface of the faceplate 102 so that they are in close proximity to the tissue 6 being imaged. In one embodiment, the first emitters 110 can emit either photoacoustic laser light or near infrared light. The first emitters 110 can emit laser light (e.g., photoacoustic wave stimulating laser light) that can be used to stimulate photoacoustic waves. The first emitters can emit photoacoustic light in addition to or instead of that emitted through the opening 114. The first emitters 110 and first detectors 112 can be disposed in any configuration, thereby allowing the imaging volume to be expanded or localized based on the number and/or spacing of first emitters 110 and first detectors 112. In one embodiment, a plurality of first emitters 110 and a plurality of first detectors 112 are disposed on opposing sides of the ultrasound transducer 104. The first emitters 110 can be disposed amongst the first detectors 112. In a similar manner, the first detectors 112 can be disposed amongst the first detectors 110.

The probe 100 can also have a plurality of second emitters 104. In an exemplary embodiment, the second emitter is an ultrasound transducer 104. In one embodiment, the ultrasound transducer is an ultrasound array 104. It will be recognized that any ultrasound array can be used in the probe 100. For example, the ultrasound array can be 1-dimensional, 2-dimensional, 1.5-dimensional or 1.75-dimensional. In an exemplary embodiment, the probe 100 can have about 1 to about 10 ultrasound transducers. A preferred number of ultrasound transducers is 1. In one exemplary embodiment, a 1-dimensional array is used.

The specific shape of the probe 100 and/or faceplate 102 is desirably configured to be an ergonomic design that is suited to traverse across the tissue 160 of a patient without causing discomfort to the patient (e.g., the faceplate 102 can comprise rounded edges, a smooth surface, and so forth). In addition, the probe 100 can be configured such that it can be hand held by an operator. While the exemplary depiction of the probe 100 in the FIG. 4 shows a circular cross-sectional area, the cross-sectional area can have a geometry that is square, rectangular, triangular or polygonal. In addition, it is further envisioned the probe 100 can be releasably secured to the cables 105 (e.g., fiber optic cables, wires, and so forth) that connects the probe 100 in operable communication with the source circuit 214 and detector circuit 216.

The probe 100 may also be used in an orthogonal mode as depicted in the FIG. 5. In this mode, the probe 100 has a single opening in the faceplate 102. The laser light irradiates a patient through an opening 114 that is orthogonal to the surface of the faceplate 102. As noted above, the first emitters 110 are used to irradiate the surface with near infrared radiation. The first detectors 112 detect this radiation and send it to the CPU 218 (see FIG. 1) for processing.

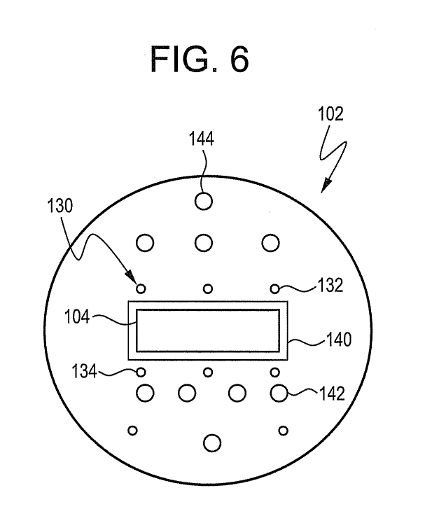

In an exemplary embodiment depicted in the FIG. 6, the faceplate 102 comprises an ultrasound transducer 104 that is disposed such that its center occupies the center of the faceplate 102. The boundaries of the ultrasound transducer 104 are surrounded by a light absorbing layer 140. In one embodiment, the boundaries of the ultrasound transducer 104 are surrounded by a band of at light absorbing layer 140. The surface area of the band of light absorbing material is substantially less than the surface area of the faceplate. In another embodiment, the entire surface of the faceplate (except those surfaces that comprise the ultrasound transducer and the first and second emitters and detectors) has disposed upon it a light absorbing layer 140.

In one embodiment, the light absorbing layer comprises black paint to absorb any reflected light. The black paint may comprise carbon black, acetylene black, carbon nanotubes, or other black colored materials that are capable of absorbing light.

In order to accommodate the differences in the DOT and PAT imaging modalities certain novelties are incorporated into the probe design to accommodate both technologies. Two of the design factors are (1) the number and placement of source and detector components and (2) the optical boundary conditions of the probe. Because of the weaker photoacoustic response, the PAT fibers have been located as close as physically possible to the transducer edges and the spacing chosen to produce the maximum fluence at the probe center for depths of 2 centimeters or greater. Fluence as defined herein is a measure of the quantity of light or other radiation falling on a surface, expressed in terms either of particles or energy per unit area.

The boundary condition of the probe, however, impacts DOT and PAT in an opposing manner. First, absorption at the probe surface due to scattered PAT illumination generates acoustic signals that propagate away from the probe, reflect off target's acoustic heterogeneities and return as signals to the transducer. These multiple reflected signals introduce significant and diffuse artifacts in the photoacoustic image. In particular, targets with a depth comparable to their extent suffer from unwanted signals appearing to originate from within the target itself. For reduced artifacts, a non-absorbing probe interface is therefore desired for PAT.

A white scattering surface (instead of a black absorbing surface) can lower the aforementioned PAT artifacts, but alters the fluences profiles for both PAT and DOT light. With increasing surface effective reflectivity, the peak of the fluence profile flattens, the peak shifts to shallower depths, and becomes higher in absolute value for all depths. This profile improves sensitivity and uniformity for PAT, but accuracy in DOT reconstruction is best with a profile that is peaked at a specific depth for a given source-detector pair separation. For highly reflective boundary conditions, the maximum imaging depth in DOT is limited to approximately 1.5 cm, preventing detection of many clinically relevant cases.

As can be seen in the FIG. 6, the photoacoustic laser light is emitted by photoacoustic fibers 130 that are disposed proximate to the boundaries of the ultrasound transducer 104. The photoacoustic fibers may include photoacoustic first emitters 132 and/or photoacoustic first detectors 134. In one embodiment, the photoacoustic fibers 130 include only photoacoustic first emitters 132 that emit laser light. The light reflected from the laser light is detected by the ultrasound transducer 104 and is processed by the CPU 218 (see FIG. 1). The photoacoustic first emitters may be present in an amount of about 3 to about 10, specifically about 4 to about 8, and more specifically about 5 to about 7.

As noted above, the probe 102 may comprise second emitters and second detectors. In the embodiment depicted in the FIG. 6, the probe 102 comprises second emitters that emit near infrared light. These second emitters are generally called DOT source fibers 142. Detectors that detect light that is reflected from the light emitted by the DOT source fibers 142 are generally called DOT detector fibers 144.

The DOT source fibers 142 and DOT detector fibers 144 are disposed further away from the boundaries of the ultrasound transducer than the photoacoustic fibers 130. In one embodiment, the average distance of all of the photoacoustic fibers 130 from the center of the faceplate 102 is less than or equal to about the average distance of the DOT source fibers 142 from the center of the faceplate 102. In another embodiment, the average distance of all of the photoacoustic fibers 130 from the center of the faceplate 102 is less than or equal to about the average distance of the DOT detector fibers 144 from the center of the faceplate 102. The DOT source fibers 142 are used to emit near infrared light from the faceplate 102 onto the surface of the patient being examined. The DOT detector fibers 144 are used to detect the light that is reflected from the surface of the patient. The reflected light is caused by the reflection of the near infrared light that is incident upon the patient from the DOT source fibers 142.

It is desirable for the surface the faceplate 102 of the probe 100 to be manufactured from an organic polymer, preferably one that is flexible at room temperature, so that it can be used to accommodate the contours of a body whose tissue is under observation. The organic polymer can comprise a wide variety of thermoplastic resins, blend of thermoplastic resins, thermosetting resins, or blends of thermoplastic resins with thermosetting resins. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, or the like, or a combination comprising at last one of the foregoing organic polymers. Exemplary organic polymers for use in the probe 100 are elastomers that have glass transition temperatures below room temperature. It is generally desirable for the organic polymer to have an elastic modulus of less than or equal to about 10⁸ pascals, specifically less than or equal to about 10⁷ pascals, and more specifically less than or equal to about 10⁶ pascals when measured as per ASTM D 638 at room temperature.

Examples of the organic polymer are polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, or the like, or a combination comprising at least one of the foregoing organic polymers.

Examples of thermosetting resins include polyurethane, natural rubber, synthetic rubber, epoxy, phenolics, polyesters, polyamides, polysiloxanes, or the like, or a combination comprising at least one of the foregoing thermosetting resins. Blends of thermosetting resins as well as blends of thermoplastic resins with thermosets can be utilized. An exemplary thermosetting resin is polydimethylsiloxane (PDMS).

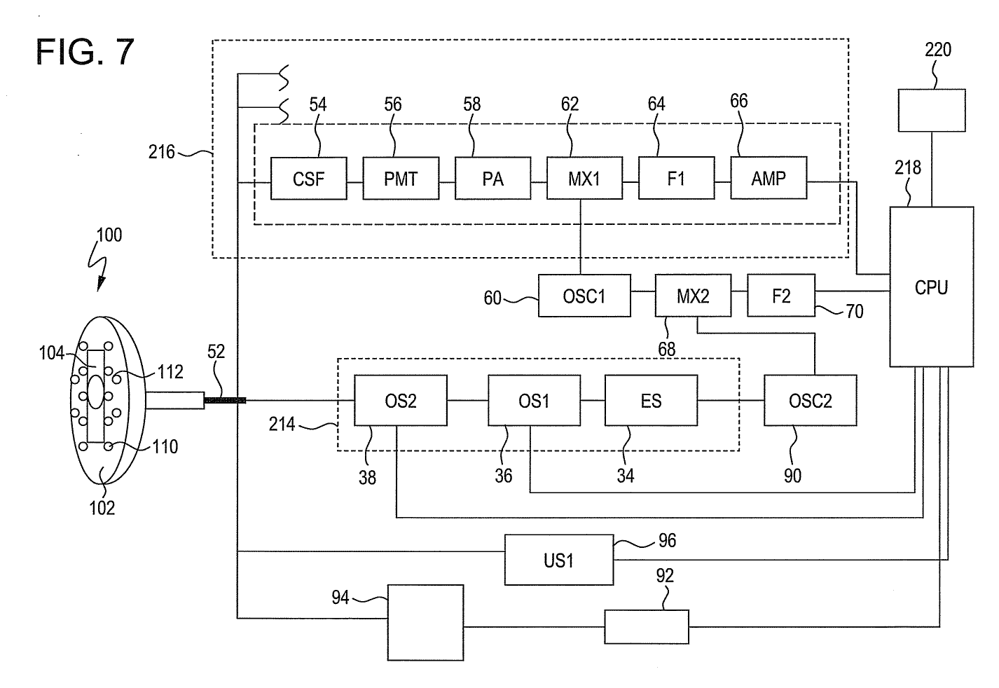

The FIG. 7 is a depiction of the circuitry used in the medical imaging apparatus. The source circuit 214 comprises an excitation source (ES) 34 that is optically connected to a primary optical switch (OS1) 36. The excitation source provides control of the near infrared radiation that is used for the DOT. The excitation source (ES) 34 comprises multiple excitation elements therein (not shown), such as pigtailed laser diodes capable of emitting near-infrared radiation at 660 nm and near-infrared radiation at 780 nanometer (nm) and 830 nm (e.g., commercially available from Thorlabs Inc.) that is modulated at a predetermined frequency (e.g., 140.00 MHz) by an oscillator (OSC2) 90, which is connected thereto.

The primary optical switch (OS1) 36 is capable of selectively connecting the emissions from any of the excitation elements, or any combination of excitation elements, to a secondary optical switch (OS2) 38 (e.g., commercially available from Piezosystem Jena Inc.). The secondary optical switch (OS2) 38 is capable of selectively directing the emissions from the primary optical switch (OS1) 36 connected to any combination of the nine emitters 10 via, hence allowing the emission of radiation through the emitters 10 selected. The emissions that are controlled by the secondary optical switch (OS2) and the primary optical switch (OS1) can be used to probe DOT as well as PAT. The primary optical switch (OS1) 36 and the secondary optical switch (OS2) 38 are connected in operable communication with, and controlled by, CPU 218.

The detectors 112 on the probe 102 are operably connected to the detector circuit 216 via optical fibers 52. The detector circuit 216 comprises detector sub-circuits 54 for each detector 212 and optically connected thereto via portions of optical fibers 52. Each detector sub-circuit 54 comprises a collimating system and filter (CSF) 54, which is capable of receiving an optical signal (e.g., light) from a detector 212, collimating the optical signal, and optionally filtering the optical signal to a specific desired frequency range. The optical signal emitted from the collimating system and filter (CSF) 54 is then directed to photomultiplier tube (PMT) 56 (e.g., commercially available from Hamamatsu Inc.) and converted into a voltage, which is subsequently amplified by pre-amp (PA) 58 (e.g., by about 40 mV). The resulting voltage is mixed with an output carrier signal having a predetermined frequency (e.g., 140.02 MHz) by a local oscillator (OSC1) 60 that is connected in electrical communication with the voltage via mixer 62. The heterodyned signals output by mixer 62 are filtered by narrowband filters (F1) 64 and further amplified (e.g., by 30 dB) by amplifier (AMP) 66. The amplified signals are then sampled at a predetermined frequency (e.g., 250 KHz) by an analog to digital conversion (A/D) board inside the CPU 218. The signals output by the oscillator (OSC1) 60 are directly mixed with the output of oscillator (OSC2) 90 by mixer 68 to produce a reference signal (e.g., a 20 KHz reference signal). The 20 kHz reference signal is then filtered by a narrowband filter 70 (e.g., 20 KHz) and provided as input to the CPU 218. An ultrasound generator and analyzer 96 is in communication with the probe 100 and produces the ultrasound waves to probe and analyzer tumors or lesions in the patient.

Lasers can emit radiation that is used to generate photoacoustic waves in a lesion or in a tumor. The acoustic waves are then imaged using the ultrasound imaging system 96 to provide an accurate location of the tumor or the lesion in the patient. The laser is a Ti:Sapphire (Symphotics TII, LS-2134) laser 94 optically pumped with a Q-switched Nd:YAG laser (Symphotics-TII, LS-2122) 92 that delivers 8 to 12 nanosecond pulses with energies up to 40 millijoules at 15 hertz (Hz) in the 700 to 830 nanometer wavelength range. The beam was subjected to divergence with a plano-concave lens to produce a uniform illumination at the surface of the faceplate 102. The radiance at the sample was below 15 mJ/cm² for all experiments. The incident beam is generally about 20 millimeters. As noted above, two different PAT configurations can be used. One is depicted in the FIG. 4. In the first (reflection mode) implementation, the laser beam was expanded to about 1 cm diameter and directed at an oblique angle at the side of a 3.5 MHz, 64-channel ultrasound transducer located at the center of the hybrid probe. The laser beam can also be incident through optical fibers as shown in FIG. 3. The photoacoustic signals can also be received by an annular transducer array or a linear array with an imaging plane parallel to the incident beam.

In the second (orthogonal mode) geometry, a Nd:YAG pumped Ti:Sapphire laser operating at 780 nm and up to 30 mJ is expanded and turned with a mirror to produce a incident diameter of about 20 mm through the central hole of the hybrid probe. The laser beam can also incident through optical fibers 105 as shown in FIG. 3. The photoacoustic signals are received by an annular transducer array (concave or convex) or a linear array with an imaging plane orthogonal to the incident beam and parallel to the depth-based DOT imaging cross-sections.

For both probe geometries, the detected ultrasound signals following the laser pulse were digitized and the optical contrast imaged using delay-and-sum and exact back projection algorithms.

In one embodiment, in one manner of using the apparatus, the probe is moved over the skin of a patient. Near infrared beams, ultrasonic waves and a laser beam from the Nd:YAG pumped Ti:Sapphire laser impinge on the surface of the patient. Reflected beams are collected and processed in the CPU. Algorithms are then used to process the information following which an image is displayed on the display.

The invention is further illustrated by the following non-limiting examples.

### EXAMPLES

### Example 1

This example was conducted to demonstrate the synergistic role of PAT and DOT in detection and characterization of deep, closely spaced targets. Because both PAT and DOT utilize optical contrast, this guidance can be more specific than with non-optical modalities to improve reconstruction accuracy and robustness.

Three types of spherical targets embedded in turbid liquid mediums were used to simulate mechanical and/or optical contrast. Hard spherical resin balls of 1 cm diameter with (μₐ=0.07 cm⁻¹, μ′ₛ=5.5 cm⁻¹) and higher (μₐ=0.23 cm⁻¹, μ′ₛ=5.5 cm⁻¹) absorption provided a high contrast ratio of 3.3. μ_{α} is the coefficient of absorption while μ′ₛ is a reduced scattering coefficient. A pair of 1 cm-diameter near-spherical soft-gelatin absorbers of higher (μₐ=0.14 cm⁻¹, μ′ₛ=4.3 cm⁻¹) and lower (μₐ=0.08 cm⁻¹, μ′ₛ=6.32 cm⁻¹) optical contrast representative of malignant and benign lesion optical properties are used as soft tissue targets with moderate contrast (ratio=1.75). Finally, a 1 cm optically scattering silicon ball simulated fibrous lesions with low absorption but adequate mechanical contrast for ultrasound visibility for investigation of the ability of the photoacoustic technique to improve specificity of target detection.

The DOT system used for the experiments was a frequency domain imager comprising 8 pairs of dual wavelength (780 nm and 830 nm) laser diodes with their outputs coupled to the probes through optical fibers. A semi-infinite absorbing boundary condition was used in the DOT image reconstruction. On the receiving side, ten 3 mm-diameter light guides were used to couple reflected light to the photomultiplier tubes (PMTS). The light was delivered to each source position sequentially and the reflected light was detected in parallel from all PMT detectors.

The reconstruction software employed a dual-mesh inversion algorithm based upon a modified Born approximation and analytic fluence calculations under the diffusion approximation. A finer grid of 0.25×0.25×0.5 (cm³) was chosen for the target regions and a coarse grid of 1.5×1.5×1.0 (cm³) was used for the background tissue. The total imaging volume was chosen to be 9×9×4 (cm³) for all measurements. This dual-zone mesh scheme significantly reduces the total number of voxels with unknown optical properties and dramatically improves the convergence of inverse mapping of target optical properties. Image reconstructions were performed along transverse cross-sections parallel to the source/detector plane in depth increments of 0.5 cm.

For multiple targets, separate fine mesh regions were defined. The regions of interest were chosen to be twice the diameter determined from the photoacoustic images to prevent over-constraint of the inversion process and the resulting boundary distortions due to the limited number of measurement pairs and a low resolution of diffusing photons.

A Ti:Sapphire (Symphotics TII, LS-2134) laser optically pumped with a Q-switched Nd:YAG laser (Symphotics-TII, LS-2122) delivered 8-12 ns pulses at 15 Hz and 780 nm wavelength. The beam was subjected to divergence with a plano-concave lens to produce a uniform illumination at the surface of the turbid medium with submerged phantom elements. The radiance at the sample was below 15 mJ/cm² for all experiments. Photoacoustic imaging was performed in orthogonal and backward mode geometries with distinct probe designs as detailed below.

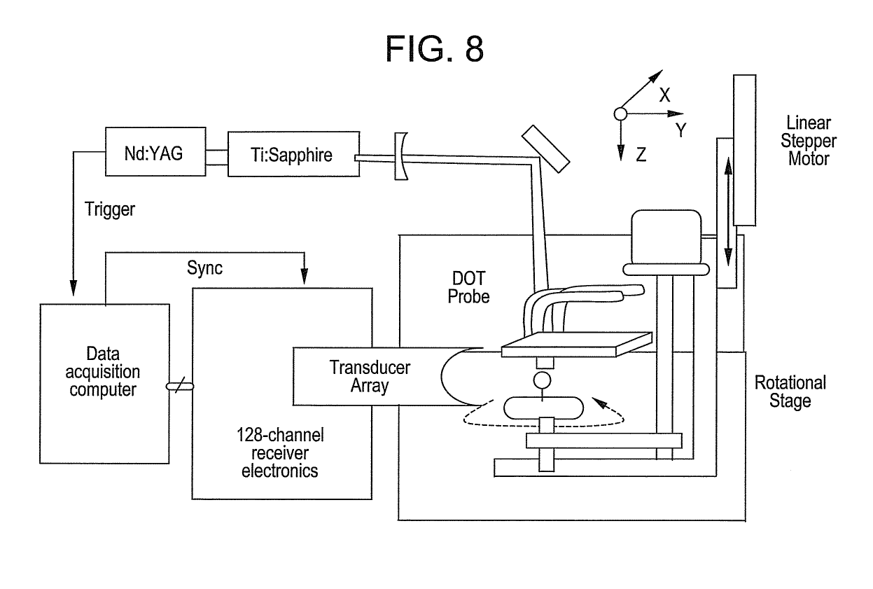

FIG. 8 depicts a picture of the experimental configuration for the orthogonal DOT/PAT geometry. For this mode, the laser light was 20 mm in diameter and positioned at the center of curvature of a 90-degree annular transducer submerged in a 50-gallon tank. The tank was filled with a 1:4 volume ratio milk/water solution and the targets submerged in the solution at various depths. The calibrated optical absorption (μₐ) and reduced scattering coefficient (μ′ₛ) of the turbid medium were in the range of 0.02 to 0.03 cm⁻¹ and 4.6 to 7.5 cm⁻¹, respectively, for this set of experiments.

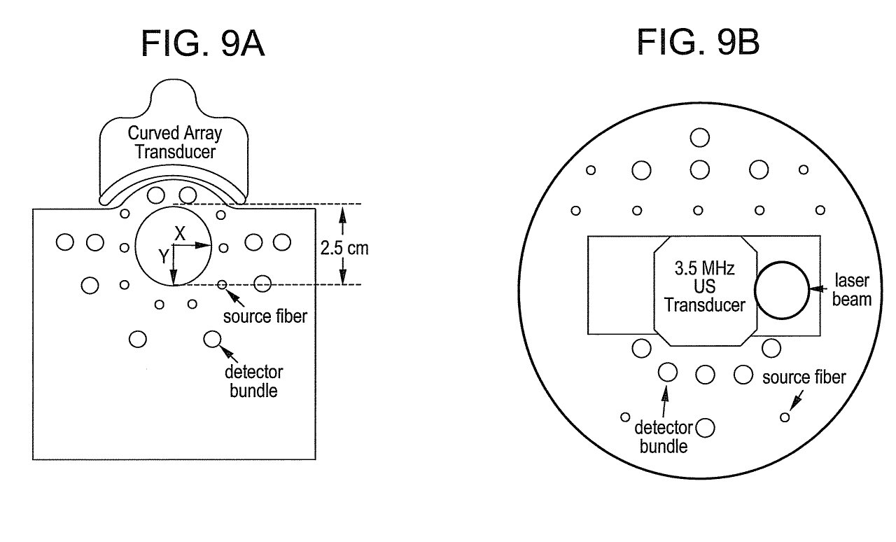

The corresponding hybrid probe is illustrated in FIG. 9. FIG. 9A reflects a probe that is used in the orthogonal mode, while FIG. 9B is used in the reflection mode. The reflection mode of the FIG. 9B is sometimes called the backward mode. The central hole of 25 mm diameter left a border around the incident beam to reduce scattered light absorption from the probe. The black absorbing boundary at the probe bottom surface, when illuminated, produces strong photoacoustic signals that propagate and reflect off the targets, introducing artifacts within the imaging region. The open region around the beam periphery was effective in minimizing these unintentional signals. The curved region of the probe mated with the corresponding surface of the transducer casing, providing registration of the PAT and DOT imaging domains.

The PAT imaging plane for this geometry was parallel to the DOT imaging planes and thus the target transverse positions and dimensions were provided directly from the PAT images. Translation of the probe and targets as a unit relative to the transducer enabled location of the central depth of the targets for the DOT mesh definitions.

The transducer comprises 128 elements arranged along a 90° arc with a 25 mm center of curvature. The center frequency of the custom piezocomposite array (Imasonic, Inc., Besancon, France) is 5 MHz with a reception bandwidth of greater than 80%. The photoacoustic signals are individually amplified up to 70 decibels (dB) and multiplexed into 16 data acquisition channels sampling at 40 MHz with 12-bit precision. Due to the multiplexing, eight laser pulses are required to generate a single 128-channel capture. The acquisition rate is ten pulses/second leading to a maximum rate of one frame/second. Images were reconstructed using the exact backprojection algorithm. To obtain a larger field of view for improved imaging, the sample was rotated three times to acquire data from 360 degrees for the PAT measurements.

For improved compatibility with the reflection DOT geometry the hybrid probe was used in a reflection-mode PAT as illustrated in FIG. 9B. In this configuration, a 1.4 cm diameter laser beam was directed at an off-axis angle of about 30 degrees for dark-field illumination underneath the transducer. A 64-channel, 3.5 MHz transducer with 60% bandwidth and 6 cm focal length captured the photoacoustic signals before digitization with a 12-bit, 50 MHz acquisition system. Due to the poor sensitivity of the transducer, the data was averaged 64 times to obtain an improved signal-to-noise ratio.

Multi-lobed tumors present a formidable challenge for accurate quantification with DOT due to the nonlinear and diffuse interactions of the photon density waves with the lesions. By providing specific identification of the absorption boundaries, PAT offers the potential for discrimination of the individual lesions and improved quantification. To evaluate this advantage, pairs of the resin or gelatin balls were submerged in the milk/water solution at depths of 1.0, 1.5, and 2.0 cm. For each target depth, the center-to-center spacing was varied from approximately 1.5, 2.0, and 2.5 cm. The PAT-determined depths and sizes were directly input into the DOT mesh definition and the absorption coefficient imaged.

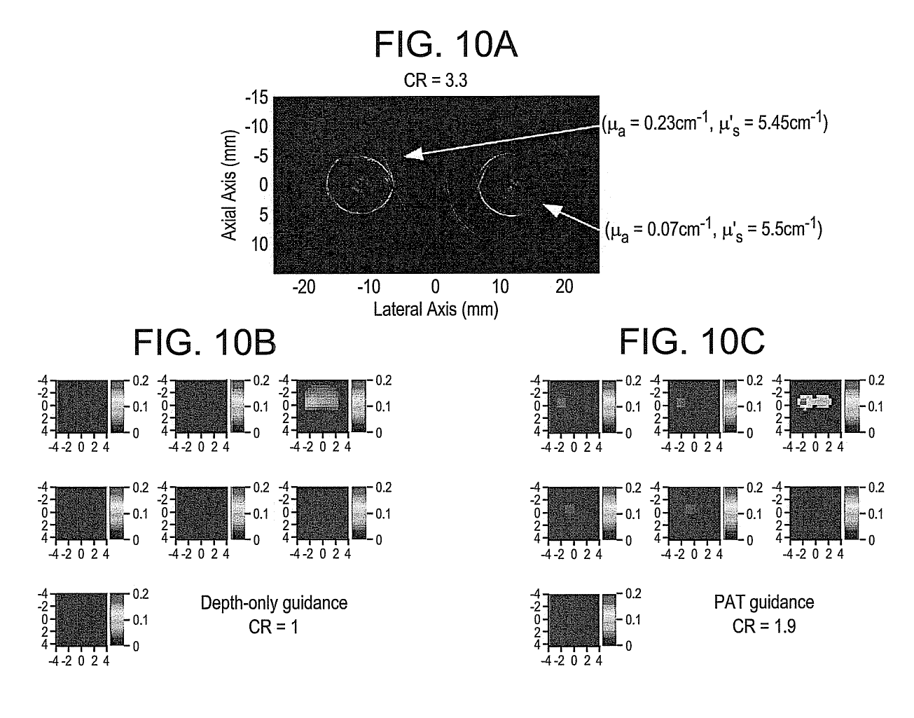

FIG. 10 depicts the PAT and DOT images for the two resin balls for a separation of 2.5 cm at a depth of 1.5 cm. FIG. 10A depicts images for the two resin balls at a separation of 2.5 cm at a depth of 1.5 cm; while FIG. 10B depicts images using depth-only guidance and FIG. 10C depicts images using PAT guidance. Because of high central frequency of the transducer and extensive low frequency electronic filtering, only the edges of the balls are visible by PAT, but this provides sufficient information for guidance of the DOT reconstruction. The corresponding DOT reconstructions, when given only the proper depth information, localize the targets to the proper cross-section but the two images are merged by a broad band. Use of the two regions-of-interest (ROI) from PAT, however, separates the two targets and clearly identifies the higher absorption ball with a contrast ratio of 1.9, closer to the true value of 3.3. The reduced contrast measured is due, in part, to the common DOT overestimation of low absorption targets and underestimation of high absorption targets.

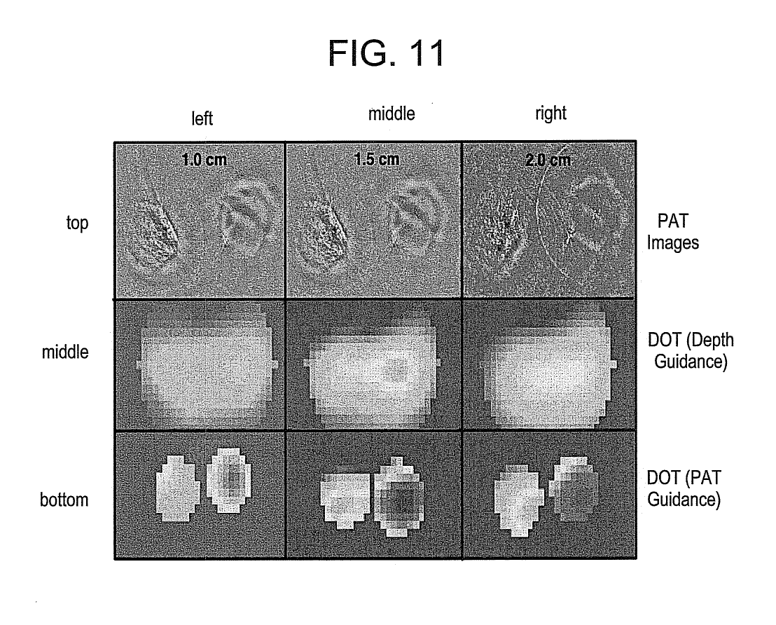

A more systematic evaluation of the potential benefits of PAT guidance was conducted using the pair of gelatin balls. FIG. 11 depicts PAT images of the two targets located at 1.0, 1.5, and 2.0 cm depths from left to right with approximately 2.0 cm target separation. The higher contrast absorber (right) shows slightly better definition of boundary than that the lower contrast absorber, however, its relative absorption map does not indicate a 1.75 higher contrast ratio. Co-registered DOT images of the same target pair reconstructed at the corresponding depth using only depth guidance are given in FIG. 11 (middle). The ROI used for DOT image reconstruction is 6 cm in diameter. The two targets were barely visible at 1 and 1.5 cm depths and merged together at the 2 cm depth. The reconstructed maximum values were only 53% to 70% of the higher contrast absorber value.

When the target center location and size equal to twice the target diameter (2.2 cm) measured from PAT images were used to segment the entire x-y plane into two ROIs for DOT reconstruction, the two targets were resolved well (FIG. 11 (bottom)). With PAT guidance the reconstructed maximum absorption coefficients of higher and lower contrast targets were 0.12 cm⁻¹ (86% of the true value) and 0.092 cm⁻¹ (113%) at 1 cm depth; 0.152 cm⁻¹ (109%) and 0.103 cm⁻¹ (129%) at 1.5 cm depth; and 0.139 cm⁻¹ (99%) and 0.104 (130%) at 2.0 cm depth. The contrast ratio (CR), defined as the ratio of the maximum absorption coefficients of the higher and lower contrast absorbers, was 1.1 at 1 cm depth, 1.1 at 1.5 cm depth, and not measurable at 2.0 cm depth. This ratio was significantly improved to 1.3, 1.5, and 1.4 at 1.0, 1.5, and 2.0 cm depths with the PAT guidance (bottom)).

Table 1 presents the reconstructed maximum absorption coefficients obtained from different target depths and separations. The target center-to-center distance used for DOT image reconstruction (DOT-d) and measured by PAT (PAT-d) is also given for each experiment. The average ratio of DOT-d/PAT-d was 1.08 with a standard deviation of 0.1. When the two targets were separated by 1.5 cm, the targets cannot be resolved well and reconstructed correctly even with PAT guidance. The cross coupling of the scattered waves generated from closely located targets is more pronounced when these targets are closer to each other. However, the higher contrast absorber was improved from 57% to 70% to 81% to 96% with PAT guidance for the conditions indicated by the shaded cells. Limited improvement was achieved for shallow depths because the large 25 mm opening introduced for the PAT beam prevents close location of the source detector DOT pairs required for good imaging at such depths.

TABLE 1

| | Target Separation | Target Separation | Target Separation |
|---|---|---|---|
| Reconstructed μₐ | Approx. 1.5 cm | Approx. 2.0 cm | Approx. 2.5 cm |
| At target-depth 1.0 | | | |
| Higher | 0.113 cm⁻¹ (81%) | 0.120 cm⁻¹ (86%) | 0.115 cm⁻¹ (86%) |
| Lower | 0.110 cm⁻¹ (138%) | 0.092 cm⁻¹ (113%) | 0.087 cm⁻¹ ((109%) |
| Contrast-ratio CR | CR = 1.02 | CR = 1.30 | CR = 1.32 |
| DOT-d | DOT-d = 1.94 cm | DOT-d = 2.27 cm | DOT-d = 2.53 |
| PAT-d | PAT-d = 1.79 cm | PAT-d = 2.17 cm | PAT-d = 2.53 |
| At target-depth 1.5 | | | |
| Higher | 0.134 cm⁻¹ (96%) | 0.152 cm⁻¹ (109%) | 0.162 cm⁻¹ (116%) |
| Lower | 0.127 cm⁻¹ (159%) | 0.103 cm⁻¹ (129%) | 0.104 cm⁻¹ (130%) |
| Contrast-ratio CR | CR = 1.06 | CR = 1.52 | CR = 1.56 |
| DOT-d | DOT-d = 1.92 cm | DOT-d = 2.2 cm | DOT-d = 2.64 cm |
| PAT-d | PAT-d = 1.62 cm | PAT-d = 1.77 cm | PAT-d = 2.54 cm |
| At target-depth 2.0 | | | |
| Higher | 0.131 cm⁻¹ (94%) | 0.139 cm⁻¹ (99%) | 0.136 cm⁻¹ (97%) |
| Lower | 0.127 cm⁻¹ (159%) | 0.104 cm⁻¹ (130%) | 0.094 cm⁻¹ (118%) |
| Contrast-ratio CR | CR = 1.03 | CR = 1.39 | CR = 1.15 |
| DOT-d | DOT-d = 1.52 cm | DOT-d = 2.6 cm | DOT-d = 2.68 cm |
| PAT-d | PAT-d = 1.51 cm | PAT-d = 2.33 cm | PAT-d = 2.76 cm |

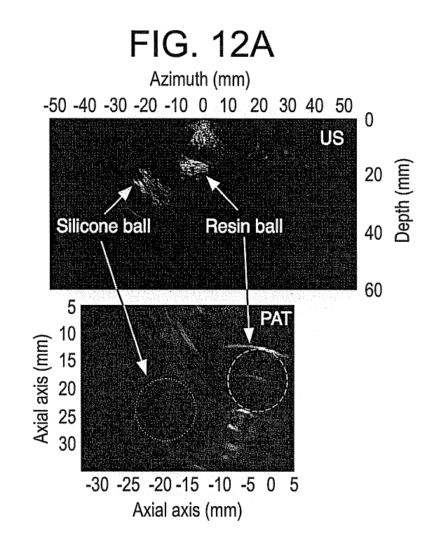

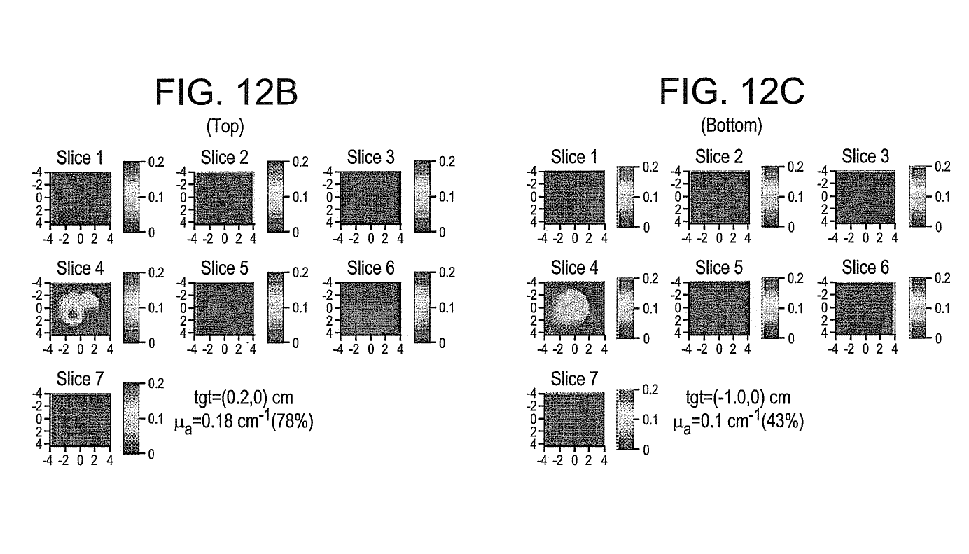

FIG. 12 depicts images using the DOT/PAT probe in the reflection mode using a resin and very low absorbing (silicone) targets at almost 2 cm depth. FIG. 12A depicts photographic images that show very low absorbing silicone targets at almost 2 cm depth.

PAT correctly images only the high absorbing target to use for PAT guidance. The limited extent of the spherical boundaries revealed by PAT is due to the small (<1.8 cm) aperture of the transducer. FIG. 12B shows DOT images using the full PAT information that allow quantification to 78% (B, top). To demonstrate the importance of precision location guidance, the central position of the guidance was displaced by 8 mm so that the target was maintained within the 2 cm diameter fine mesh ROI. Because the actual target was slightly offset from the center of the defined mesh, quantification was reduced to 43% and the resulting images were not well defined (C, bottom) as can be seen in the FIG. 12C.

### Example 2

This example was conducted to demonstrate the synergistic role of PAT and DOT in detection and characterization of deep, closely spaced targets. In this example, both single-lobed and multi-lobed polyvinylchloride (PVC) plastisol absorbers were used in separate measurements to stimulate a tumor. The PVC absorbers were disposed in Intralipid (a material that is representative of human breast tissue). The PVC absorbers were cube shaped having each side equal to 1 centimeter and had absorption coefficients of 0.075 cm⁻¹ to 0.23 cm⁻¹. The PVC absorbers were imaged at depths of up to 2.5 centimeters in the Intralipid. As will be seen in the experiment below one of the absorbers was a high contrast target and the other a low contrast target.

From the results detailed below, it can be seen that without PAT guidance the absorber location was not clear and lower contrast targets in the two-absorber configurations were not distinguishable. With PAT guidance, the two targets were well resolved and the reconstructed absorption coefficients improved to within 15% of the true values. In experiments, the cubes were submerged in Intralipid with calibrated optical parameters of (calibrated optical absorption μₐ=0.026 cm⁻¹, reduced scattering coefficient μ′ₛ=6.0 cm⁻¹) representative of breast tissue.

The absorption was conducted with a probe having a completely absorbing surface for which the reflection (Reff=0) was zero (e.g., a black surface), a partial absorbing surface (Reff=0.4) (e.g., a gray or red color absorbing surface), or a partial reflecting surface (Reff=0.6), (e.g., a white colored surface.

The system used for making measurements during the experiments was a frequency domain imager comprising 9 sets of four-wavelength (740, 780, 810, and 830 nanometers) laser diode sources with their outputs coupled to the probes through optical fibers. For the results presented herein, only the 780 nm source was used for reconstruction. All sources were modulated at 140 MHz. A semi-infinite absorbing boundary condition was used in DOT image reconstruction. On the receiving side, fourteen 3 mm diameter light guides were used to couple reflected light to the photomultiplier tubes (PMTS). The light was delivered to each source position sequentially and reflected light was detected in parallel from all PMT detectors.

A Ti:Sapphire (Symphotics TII, LS-2134) laser optically pumped with a Q-switched Nd:YAG laser (Symphotics-TII, LS-2122) delivered 8-12 nanosecond (ns) pulses at a frequency of 15 hertz (Hz) and a wavelength of 770 nanometer respectively. The laser output was expanded to approximately a 1 centimeter (cm) diameter using a Galilean telescope and spatially filtered using an iris to improve beam symmetry and reduce extraneous peripheral optical energy. The filtered beam was focused using an F#=1 lens for coupling into a custom 1×7 high-energy optical splitter assembly manufactured by OFS Specialty Photonics (Avon, Conn.).

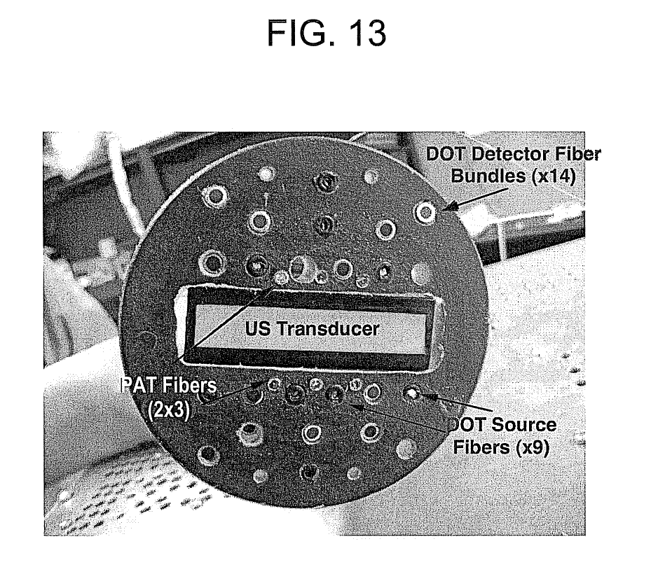

FIG. 13 is a photograph that presents a close-up view of the hybrid DOT/PAT probe. The ultrasound transducer occupies the central slot with the six PAT optical fibers (photoacoustic first emitters) straddling the transducer in a 2 row by 3-fiber configuration. The fibers, with a 2.4 centimeter (cm) spacing between the fibers across the transducer and 1 cm spacing between the fibers along the transducer, illuminate a region of approximately 2×2.5 cm. The small size and number of PAT fibers did not necessitate displacement of DOT fibers from a clinically desirable configuration. The DOT source fibers (second emitters) and detector fiber (second detector) bundles are arranged nearly symmetrically on both sides of the probe in a pattern that provides a distribution of source-detector pair distances of 1.5 to 7 cm. This organization, coupled with the black-painted absorbing probe boundary, enables optimized imaging for targets ranging from 0.5 to 2.5 cm in depth under the surface of the skin.

The assembly featured a 600-micrometer step-index input fiber with an SMA905 connector interface. The seven output fibers comprise 200-micrometer multimode fibers in a compact 2.5 millimeter (mm) stainless steel ferrule to minimize space on the probe. The seventh output fiber was used for real-time monitoring of optical energy delivered to the probe. The measured optical energy uniformity was better than 3 decibels (dB) across all outputs. The estimated transmission through the assembly was 60%. Because the output fibers have a small diameter, the input energy was restricted to less than 5 millijoules (mJ), corresponding to less than 3 mJ delivered to the six output fibers.

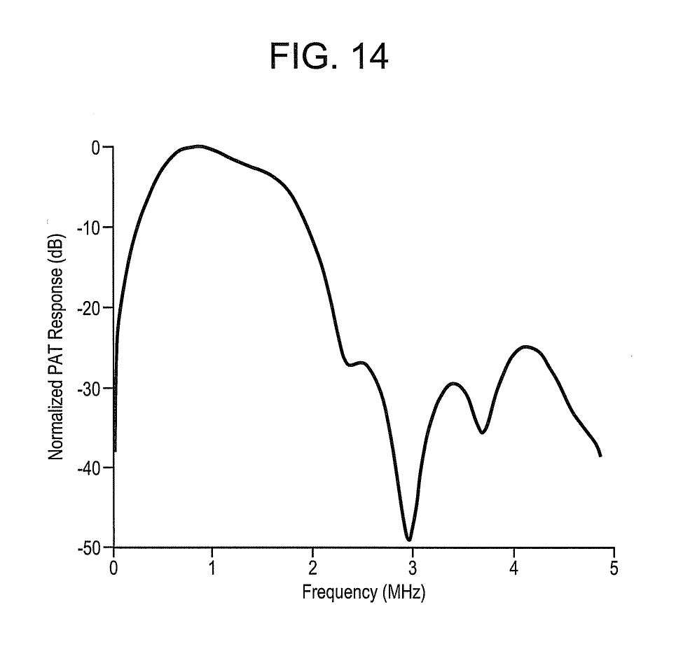

Clinically, DOT has demonstrated the capability to detect tumors of 1 cm or larger in extent. Because of the large target size, a low-frequency transducer design was employed to maximize sensitivity and more faithful imaging of features greater than 1 mm. The 1,3-piezocomposite transducer, produced by Vermon (France), contained 64 elements with 0.85 mm pitch. FIG. 14 is a plot of the photoacoustic response obtained using weakly scattered light absorbed on the transducer surface. As shown, the center frequency of the transducer is 1.3 megahertz (MHz) with a 6 dB response from 400 to 2000 kilohertz (kHz). An integrated acoustic lens with 25 mm focal length increases sensitivity at imaging depths for which the optical fluences (i.e., the number of particles that intersect a given area) are low.

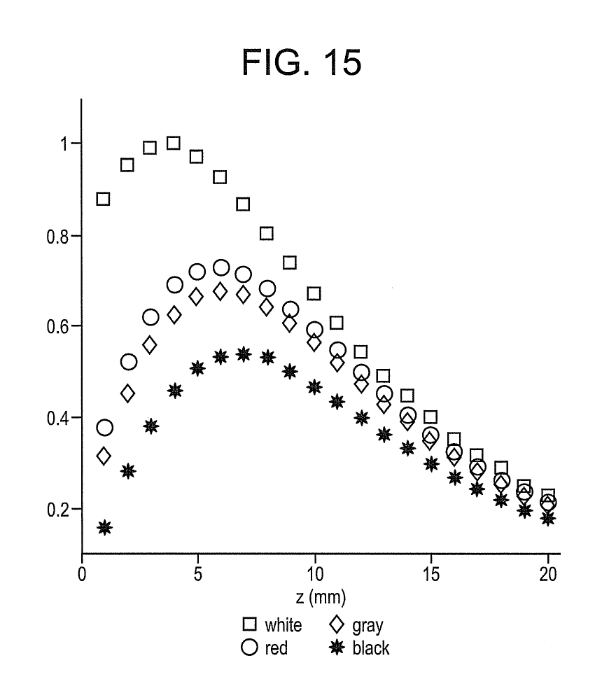

In order to evaluate the effect of boundary conditions on the fluence profiles, Monte Carlo simulation was performed using a reference configuration consisting of fibers spaced 2.8 cm across the transducer and 2 cm along the transducer. Although this spacing is larger than employed in the experimental probe, the conclusions remain valid. FIG. 15 depicts the profiles in the central region of the field vs. depth (z) for absorbing (Reff=0, black color) and moderately absorbing (Reff=0.4, gray and red color) and partial reflective (Reff=0.5, white) probe surfaces respectively. The simulations were validated by experiments and show that more than 20% increase in fluence was obtained with the partial reflective probe.

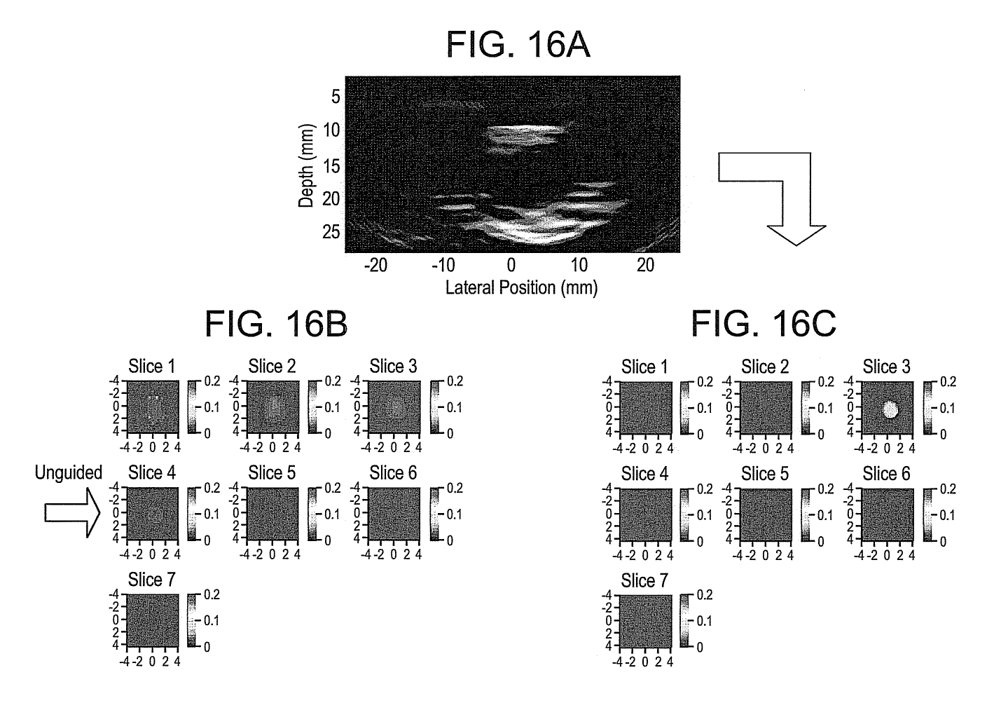

FIG. 16 shows the improvement in both DOT localization and quantification provided with PAT guidance. FIG. 16A is a photograph showing depth of the absorber versus lateral position traversed across the surface with the probe. FIG. 16B shows photographs that depict the image obtained using only the DOT probe for the single-lobed inclusion. FIG. 16C shows photographs that depict the image obtained using both the DOT and the PAT probe for the single-lobed inclusion.

Without specific identification of the target location or size, the image is diffuse and spread over multiple depths with a low maximum reconstructed value of 0.05 as may be seen in the FIG. 16B. With PAT localization, the absorption was correctly isolated to the appropriate depth and the value increased to 0.095 for the 0.075 cm⁻¹ target as may be seen in the FIG. 16C.

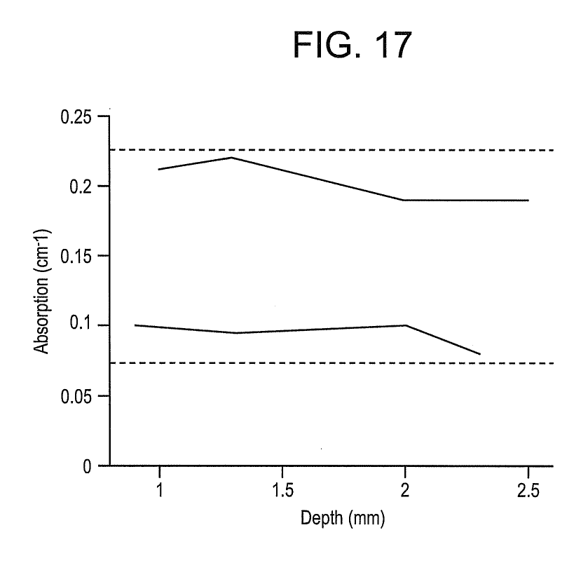

To better quantify the accuracy of the PAT-guided DOT, the high-contrast and low contrast targets were located at depths of 1.0, 1.5, 2.0, and 2.5 cm. FIG. 17 is a graph that presents the reconstructed absorption values versus depth. The quantified value fairly constant over the entire depth range although a slight falloff is observed. Generally, DOT quantifications are known to produce overestimates of low-contrast targets and under-estimation for high-contrast targets in agreement with the measurements. The dashed lines were true values and solid line represents the reconstructed absorption of the high-contrast target at the different depths (1.0, 1.5, 2.0, and 2.5 cm) listed above and blue line shows the reconstructed absorption of the low-contrast target at the different depths listed above. The overall agreement to within 20% over the entire depth range demonstrates the potential of the combined DOT/PAT approach.

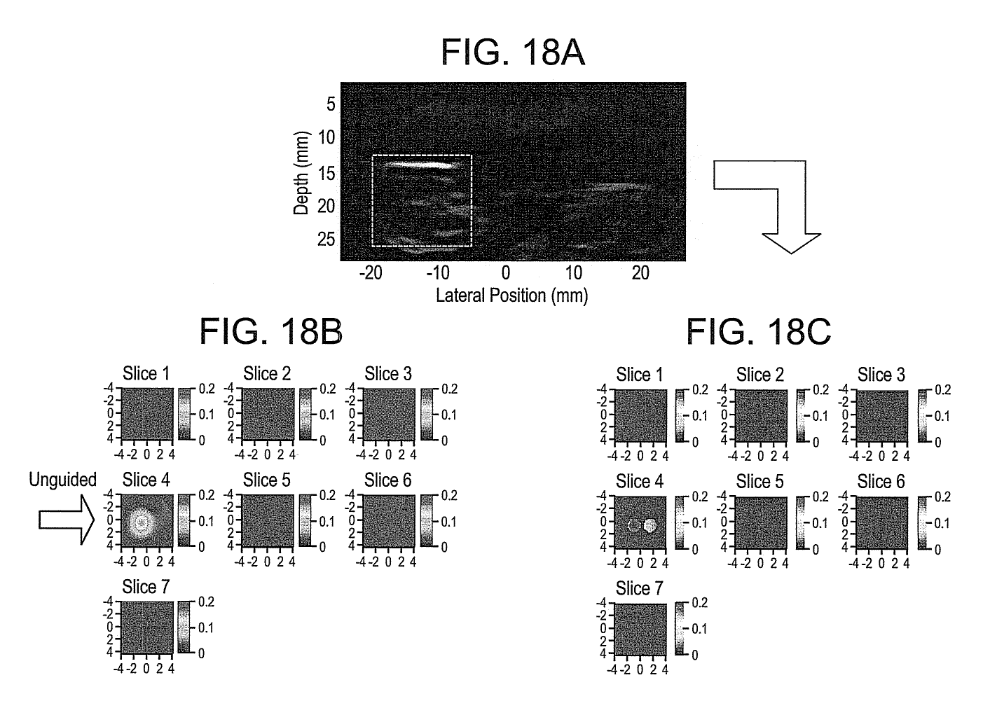

Multi-lobed tumors present a formidable challenge for accurate quantification with DOT due to the nonlinear and diffuse interactions of the photon density waves with the lesions. By providing specific identification of the absorption boundaries, PAT offers the potential for discrimination of the individual lesions and improved quantification. To demonstrate this advantage, the high-contrast and low-contrast targets were spaced by 2.0 cm at a depth of 2.0 cm. FIG. 18 shows the reconstructed absorbers with and without PAT guidance. FIG. 18A is a photograph showing depth of the absorber versus lateral position traversed across the surface with the probe. FIG. 18B shows photographs that depict the image obtained using only the DOT probe for the multi-lobed inclusion. FIG. 18C shows photographs that depict the image obtained using both the DOT and the PAT probe for the multi-lobed inclusion. From the FIG. 18B, it may be seen that in the absence of optical guidance, the high-contrast absorber (left) was localized but the low-contrast absorber (right) was not visible. As a result, the reconstructed value was intermediate between the true absorption coefficients. From the FIG. 18C, it may be seen that the PAT guidance enabled separation of the distinct regions and quantification of each absorber to within 10%, faithfully determining the contrast ratio of over 2.

This study demonstrates the synergistic role of PAT and DOT in detection and characterization of deep, closely spaced targets. Because both PAT and DOT use optical contrast, this guidance can be more specific than with conventional non-optical modalities to improve reconstruction accuracy and robustness. This can be particularly important for more complex absorption profiles such as the clustered tumors or closely spaced lymph nodes observed in clinical environments. As optical absorption changes are directly related to tumor angiogenesis process, this hybrid technology has a great potential for simultaneous cancer detection and diagnosis.

While this initial demonstration of a clinical probe design was focused on validating the robustness and improvements in DOT quantification with PAT serving as an adjunct modality, the ultimate goal of the system is to exploit the advantages of both technologies for cancer detection and diagnostics. PAT can provide high-resolution imaging of lesion heterogeneity, especially when aided by DOT determination of background optical parameters. The DOT determination of background optical parameters enables the introduction of an optical illumination model for more accurate conversion of absorbances (fluence×absorption coefficient) to the clinically relevant absorption coefficient (or equivalently hemoglobin concentration). Similarly, DOT can provide coarse 3D images of optical absorption while offering absolute quantification of volumetric chromophore concentrations when assisted by PAT guidance.

Photoacoustic guidance of DOT is shown to enable nearly depth-independent quantification of single targets as well as delineation and independent quantification for heterogeneous absorption features. The synergistic combination of both optical modalities offers the promise of both high-resolution and absolute quantification of total hemoglobin for cancer detection and diagnosis.

This study demonstrates the synergistic role of PAT and DOT in detection and characterization of deep, closely spaced targets. Because both PAT and DOT utilize optical contrast, this guidance can be more specific than with conventional non-optical modalities to improve reconstruction accuracy and robustness. This can be particularly important for more complex absorption profiles such as the clustered tumors or closely spaced lymph nodes observed in clinical environments. As optical absorption changes are directly related to tumor angiogenesis process, this hybrid technology has a great potential for simultaneous cancer detection and diagnosis. The current implementation of the PAT and DOT systems can be further improved to address clinical applications. For example, the reflection-mode transducer can be replaced with a larger aperture, wideband, low-frequency transducer array to improve boundary definition and discrimination of relative contrast among multiple targets. The lower ultrasound transducer frequency also enhances detection of low frequency components from deeper and larger lesions. In addition, light delivery from an array of fibers along the transducer axis can produce more uniform illumination under the transducer and enable modeling of the fluence profiles for more accurate PAT imaging.

While the invention has been described in detail in connection with a number of embodiments, the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method for medical imaging comprising:
scanning a tissue volume with a near-infrared photoacoustic laser beam to obtain a first set of structural parameters, wherein the tissue volume includes a biological entity;
receiving from the tissue an acoustic signal in response to scanning the tissue volume with the laser beam; the acoustic signal being processed to obtain a first set of structural parameters;
scanning the tissue with ultrasonic waves to obtain a second set of structural parameters;
scanning the tissue with near-infrared diffusive light to obtain a third set of structural parameters; and
processing the first and second sets of structural parameters and localizing the biological entity using these parameters to quantitatively reconstruct the functional parameters of the biological entity from the third set of structural parameters.

2. The method of claim 1, wherein the biological entity comprises a tumor or a lesion.

3. The method of claim 1, wherein the photoacoustic laser is a Q-switched titanium:sapphire laser delivering 8 to 12 nanosecond pulses with energies up to 40 millijoules.

4. The method of claim 1, wherein the near infrared photoacoustic laser is a gas laser, a solid state laser and/or a diode laser.

5. The method of claim 1, wherein the near-infrared diffusive light is obtained from a laser diode.

6. The method of claim 1, wherein the structural parameters provide structural information and/or functional information about the biological entity contained in the scanned volume.

7. The method of claim 1, wherein the photoacoustic signal is used to obtain structural information about biological entities that are about 2 to about 3 centimeters under a patient's skin.

8. The method of claim 1, wherein the first set of structural parameters and the second set of structural parameters can be used to obtain information about biological entities that are located about 1 to about 5 centimeters under a patient's skin.

9. An apparatus for biological imaging comprising:
a probe comprising an emitter and a detector;
a source circuit connected in operational communication to the emitter;
a detector circuit connected in operational communication to the detector;
a central processing unit connected to the source circuit and the detector circuit;
a display operably connected to the central processing unit; and,
wherein the apparatus is operative to perform photoacoustic tomography and diffusive optical tomography and/or ultrasound tomography.

10. The apparatus of claim 9, wherein information obtained from the ultrasound tomography is combined with information obtained from photoacoustic tomography and diffusive optical tomography.

11. The apparatus of claim 9, wherein the photoacoustic tomography is obtained by using a laser that comprises a titanium:sapphire laser optically pumped with a Q-switched Nd:YAG laser that delivers 8 to 12 nanosecond pulses at 15 hertz.

12. The apparatus of claim 9, wherein the photoacoustic tomography is obtained by using a near infrared photoacoustic laser; the near infrared photoacoustic laser beings a gas laser, a solid state laser and/or a diode laser.

13. The apparatus of claim 9, wherein the probe comprises a faceplate having a first surface and a second surface; the first surface being opposed to the second surface; the faceplate having openings for accommodating a plurality of first emitters and second emitters; an ultrasound transducer; the ultrasound transducer being disposed in the faceplate and having a surface that is parallel to the first surface of the faceplate; a perimeter of the ultrasound transducer being surrounded by light absorbing material; first emitters; and second emitters; wherein the first emitters are closer to a center of the faceplate than the second emitters.

14. A probe comprising:
a faceplate having a first surface and a second surface; the first surface being opposed to the second surface; the faceplate having openings for accommodating a plurality of first emitters and second emitters;
an ultrasound transducer; the ultrasound transducer being disposed in the faceplate and having a surface that is parallel to the first surface of the faceplate; a perimeter of the ultrasound transducer being surrounded by light absorbing material;
first emitters; and
second emitters; wherein the first emitters are closer to a center of the faceplate than the second emitters.

15. The probe of claim 14, further comprising a plurality of first detectors and second detectors.

16. The probe of claim 14, wherein the first emitters and the second emitters are optical fibers that have an end disposed in the faceplate.

17. The probe of claim 15, wherein the second emitters and the second detectors are optical fibers that have an end disposed in the faceplate.

18. The probe of claim 14, wherein the faceplate has a cross-sectional area that is circular.

19. The probe of claim 14, wherein the faceplate comprises an elastomer.

20. The probe of claim 14, wherein the perimeter of the ultrasound transducer is surrounded by a band of light absorbing material; the surface area of the band of light absorbing material being substantially less than the surface area of the faceplate.

21. The probe of claim 14, comprising about 3 to about 10 first light emitters.

22. The probe of claim 14, wherein the second light emitters emit near infrared radiation of about 600 nanometers to about 100 micrometers.

23. The probe of claim 14, wherein the ultrasound transducer is concentrically arranged with respect to the faceplate.

24. The probe of claim 14, wherein the probe can be used in the orthogonal mode or in the reflection mode.
